(19) United States
(12) Patent Application Publication
(10) Pub. No.: US 2008/0188653 A1
(43) Pub. Date: Aug. 7, 2008

(54) Process for Preparation of Mycophenolate Mofetil

(75) Inventors: Mei-Jing Lee, Taoyuan (TW); Rung-Tian Suen, Taoyuan (TW); Yu-Liang Liu, Taoyuan (TW); Ching-Peng Wei, Taoyuan (TW)

Correspondence Address: WPAT, PC;INTELLECTUAL PROPERTY ATTORNEYS, 2030 MAIN STREET, SUITE 1300, IRVINE, CA, 92614, US

(73) Assignee: FORMOSA LABORATORIES, INC., Taoyuan (TW)

(21) Appl. No.: 11/670,990

(22) Filed: Feb. 4, 2007

(51) Int. Cl. C07D 413/12

(52) U.S. Cl. 544/153

(57) ABSTRACT

The present invention provides a novel process for the preparation of mycophenolate mofetil, by heating mycophenolic acid with 2-morpholinoethanol in an organic solvent in the presence of drying agent.

### FIELD OF THE INVENTION

The present invention relates to a process for the preparation of mycophenolate mofetil.

### BACKGROUND OF THE INVENTION

Mycophenolate mofetil is the morpholinoethyl ester of mycophenolic acid and has the formula as following.

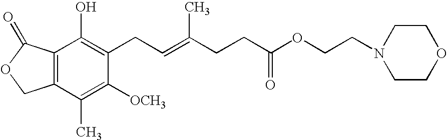

Mycophenolic acid (MPA) is the first well characterized antibiotic. In addition to its antibiotic activity, it also has antifungal, antiviral, antitumor, and immunosuppression properties. MPA was withdrawn due to its high incidence of side effects. Mycophenolate mofetil (CellCept.®), the 2-morpholinoethyl ester derivative of MPA, does not have these drawbacks, and has a better bioavailability than mycophenolic acid. After oral administration the ester form rapidly hydrolyzes to free acid of MPA. Mycophenolate mofetil, the pharmaceutically acceptable salts thereof, and the immunosuppressive, anti-inflammatory, anti-tumor and anti-viral uses thereof are described in U.S. Pat. No. 4,753,935, incorporated herein by reference. As immunosuppressive agents, the Mycophenolate mofetil is useful in treating auto-immune related disorders, glomerulonephritis and hepatitis, and in preventing allograft rejection. As anti-inflammatory agents, it is useful in treating rheumatoid arthritis. As anti-tumor agents, it is useful in treating solid tumors and malignancies of lymphoreticular origins.

The production of mycophenolate mofetil from the fermentation product of mycophenolic acid and 2-morpholinoethanol is a demanding procedure because of the basic function of the morpholine moiety and the polyfunctionality of the mycophenolic acid. One known process comprises, for example, esterification without a catalyst with relatively long reaction times. Other known processes for the production of mycophenolate mofetil via condensation between the MPA and the N-(2-hydroxyethyl)morpholine by means of the acyl chloride of MPA or by using a coupling reagent, such as dicyclohexylcarbodiimide (DCC). However, the conventional methods are regarded as having little suitability or none at all for producing mycophenolate mofetil in pharmaceutically acceptable purity, especially because of impurities that arise. In order to overcome the problem, modifying process with high purity and high yield was disclosed in EP 1667987 and US 2005250773. Other alternative methods such as making mycophenolate mofetil by transesterification or using a biocatalytic method are disclosed in US 20040167130, WO 2006/024582, and WO 00/34503.

Those methods mentioned above require the use of a catalyst to achieve acceptable yields. However, catalytic reactions entail the added cost of the catalyst and the additional steps of its addition and separation from the reaction mixture. Otherwise, operation with rigorous control of the experimental conditions is a disadvantage of the biocatalytic method with respect to industrial application. Thus, a non-catalytic alternative for synthesizing mycophenolate mofetil has been desired. It has been disclosed in U.S. Pat. No. 5,247,083, WO 94/01427, and WO 02/100855 that good yields of mycophenolate mofetil can be obtained without the disadvantage of the prior described methods and without the use of a catalyst. Moreover, the reaction of mycophenolic acid and 2-morpholinoethanol gives at least the equivalent or increased yields by omitting the use of a catalyst.

However, inert organic solvents are needed in the non-catalytic method mentioned above for azotropic removal of water generated by the reaction. Reaction condition changes along with different solvent and the reaction mixture need to be warmed up to boiling under azotropic separation of water.

### SUMMARY OF THE INVENTION

The present invention provides a novel and efficient process for the non-catalytic preparation of mycophenolate mofetil, by heating mycophenolic acid with 2-morpholinoethanol in an organic solvent in the presence of drying agent. It has surprisingly been discovered that the water can be removed easily just by the usage of drying agent without by means of azotropic distillation under high-boiling solvent. Also, the reaction is slightly accelerated and the product is prepared with high purity. It is more convenient and efficient with respect to industrial application. The present invention is described in greater detail below in the Summary and Detailed Description of the Invention.

### BRIEF DESCRIPTION OF THE DRAWINGS

FIG. 1 shows the preparation of the present invention.

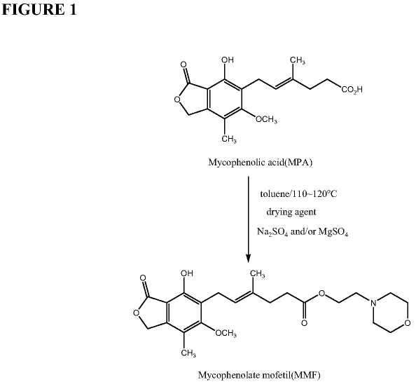

### DETAILED DESCRIPTION OF THE INVENTION

The present invention provides a novel and efficient process for the preparation of mycophenolate mofetil, by heating mycophenolic acid with 2-morpholinoethanol in an organic solvent in the presence of drying agent capable of removal of water with respect to industrial application.

Accordingly, mycophenolic acid is esterified slowly by heating with slightly excess 2-morpholinoethanol in an organic solvent in the presence of drying agent selected from the group consisting of alkali earth sulfate, alkaline earth sulfate, and alkaline earth halide, preferably selected from sodium sulfate and magnesium sulfate. The organic solvent is selected from the group consisting of ketone, aromatic hydrocarbon, ester, ether, nitrile, and halogenated hydrocarbon, preferably is selected from toluene and xylene, more preferably is toluene.

In the present invention, the reaction mixture is heated until the pot temperature is ranging from about 80° C. to 155° C., preferably from about 95° C. to 135° C., more preferably from about 110° C. to 120° C. The reaction temperature is kept at this range for performing the condensation reaction in a period of about 6 to 48 hours, more preferably about 6 to 30 hours until completion of reaction. When mycphenolate mofetil (checked by HPLC) is formed, the desired product is obtained by cooling, extraction, washing, crystallization, centrifuge, recrystallization, centrifuge and dried under vacuum with purity over 99.5%. In the preferred embodiment, the recrystallization is made from an organic solvent which is selected from the group consisting of ketone, ester, and aromatic hydrocarbon, more preferably is selected from methyl isobutyl ketone, ethylacetate, xylene, and toluene, most preferably is ethylacetate.

Isolation and purification of the compound described herein can be effected. If desired, any suitable separation or purification procedure such as filtration, extraction, crystallization, column chromatography, thin-layer chromatography or thick-layer chromatography, or a combination of these procedures can be applied in the present invention. Specific illustrations of suitable separation and isolation procedures can be made by reference to the examples herein. However, other equivalent separation or isolation procedures can, of course, also be used.

### EXAMPLE

The examples below are non-limited and are merely representative of various aspects and features of the present invention.

### Example 1

### Preparation of Mycophenolate Mofetil: Use of Sodium Sulfate as Drying Agent

In a 50 L of stainless reactor, 11.56 Kg of toluene, 10.0 Kg of mycophenolic acid (MPA), 6.14 Kg of 2-morpholinoethanol, and 1.0 Kg of sodium sulfate were added. The reaction mixture was heated until the pot temperature was between 110° C. and 120° C. The reaction temperature was kept at this range for performing the condensation reaction in a period of 24 hours.

When mycphenolate mofetil was formed, the reaction mixture was cooled below 100° C. and added with 11.0 Kg of toluene and 10.0 Kg of water. The reaction mixture was maintained between 50° C. and 60° C., and further vigorously agitated for 10˜15 minutes. The layers were separated and organic layer was washed with 5% sodium bicarbonate aqueous solution. The organic layer was added with 10.0 Kg of water and further vigorously agitated for 10˜15 minutes. The aqueous layer was discarded from this reactor. The reaction mixture was cooled to 0˜10° C. and kept stirred about 8 hours. 9.2 Kg of white crystalline powder was obtained by centrifuge. Loss on drying of this wet cake was about 12.8%. Purity by HPLC was 99.6%.

9.2 Kg of wet cake were put in a 50 L of reactor with a reflux condenser together with 14.4 Kg of ethylacetate. The reaction mixture was heated to about 52° C. The resulting solution was passed through the filter into another 50 L of stainless reactor, and then was cooled to 20˜30° C. about 4 hours. The solution was further cooled to 0˜10° C. and kept stirred not less than 8 hours. 8.0 Kg of white crystalline powder was obtained by centrifuge. 7.2 Kg of pure title compound was obtained by vacuum dried. (Purity by HPLC: 99.9%, M.P. by DSC: 95.7° C.)

### Example 2

### Preparation of Mycophenolate Mofetil: Use of Magnesium Sulfate as Drying Agent

In a 50 L of reactor, 13.0 Kg of toluene, 10.0 Kg of mycophenolic acid (MPA), 4.5 Kg of 2-morpholinoethanol, and 1.0 Kg of magnesium sulfate were added. The reaction mixture was heated until the pot temperature was between 110° C. and 120° C. The reaction temperature was kept at this range for performing the condensation reaction within 24 hours.

When mycphenolate mofetil was formed, the reaction mixture was cooled below 100° C. and added with 9 Kg of toluene and 10 Kg of water. The reaction mixture was maintained between 50° C. and 60° C., and further vigorously agitated for 10˜15 minutes. The layers were separated and organic layer was washed with 5% sodium bicarbonate aqueous solution. The organic layer was added with 10 Kg of water and further vigorously agitated for 10˜15 minutes. The aqueous layer was discarded from this reactor. The reaction mixture was cooled to 0˜10° C. and kept stirred about 8 hours. 9.0 Kg of white crystalline powder was obtained by centrifuge. Loss on drying of this wet cake was about 10.0%. Purity by HPLC was 99.6%.

9.0 Kg of wet cake were put in a 50 L of stainless reactor with a reflux condenser together with 14.6 Kg of ethylacetate. The reaction mixture was heated to about 52° C. The resulting solution was passed through the filter into another 50 L of stainless reactor, and then was cooled to 20˜30° C. about 4 hours. The solution was further cooled to 0˜10° C. and kept stirred not less than 8 hours. 8.1 Kg of white crystalline powder was obtained by centrifuge. 7.3 Kg of pure title compound was obtained by vacuum dried (Purity by HPLC: 99.7%, M.P. by DSC: 95.3° C.).

The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claim

What is claimed is:

1. A process for preparing mycophenolate mofetil by heating mycophenolic acid with 2-morpholinoethanol in an organic solvent in the presence of drying agent.

2. The process according to claim 1, wherein the drying agent is selected from the group consisting of alkali earth sulfate, alkaline earth sulfate, and alkaline earth halide.

3. The process according to claim 2, wherein the drying agent is selected from sodium sulfate and magnesium sulfate.

4. The process according to claim 1, wherein the organic solvent is selected from the group consisting of ketone, aromatic hydrocarbon, ether, ester, nitrile, and halogenated hydrocarbon.

5. The process according to claim 4, wherein the organic solvent is selected from toluene and xylene.

6. The process according to claim 5, wherein the organic solvent is toluene.

7. The process according to claim 1, wherein the reaction temperature is ranging from about 80° C. to about 155° C.

8. The process according to claim 7, wherein the reaction temperature is ranging from about 95° C. to about 135° C.

9. The process according to claim 8, wherein the reaction temperature is ranging from about 110° C. to about 120° C.

10. The process according to claim 1, wherein the reflux time is about 6 to 48 hours.

11. The process according to claim 10, wherein the reflux time is about 6 to 30 hours.

12. The process according to claim 1, further comprising extraction and purification of the mycophenolate mofetil by cooling, extraction, washing, crystallization, centrifuge, recrystallization, centrifuge, and dried under vacuum.

13. The process according to claim 12, wherein the recrystallization is made from an organic solvent.

14. The process according to claim 13, wherein the organic solvent is selected from the group consisting of ketone, ester, and aromatic hydrocarbon.

15. The process according to claim 14, wherein the organic solvent is selected from the group consisting of methyl isobutyl ketone, ethylacetate, xylene, and toluene.

16. The process according to claim 14, wherein the organic solvent is toluene.
